(19) United States
(12) Patent Application Publication
(10) Pub. No.: US 2025/0307497 A1
(43) Pub. Date: Oct. 2, 2025

(54) METHOD AND DEVICE FOR DESIGNING CONDUCTIVE CATHODE MATERIAL BASED ON MACHINE LEARNING

(71) Applicant: Foundation of Soongsil University-Industry Cooperation, Seoul (KR)

(72) Inventors: Kyoungmin Min, Suwon-si (KR); Jinyoung Jeong, Seoul (KR)

(73) Assignee: Foundation of Soongsil University-Industry Cooperation, Seoul (KR)

(21) Appl. No.: 18/789,690

(22) Filed: Jul. 31, 2024

(30) Foreign Application Priority Data
Apr. 2, 2024 (KR) 10-2024-0044610

(51) Int. Cl. G06F 30/27; G06F 119/06

(52) U.S. Cl. CPC G06F 30/27; G06F 2119/06

(57) ABSTRACT

A method for designing a cathode material includes determining whether a first electrode material corresponds to a first cathode material candidate, based on a cathode material candidate filtering model, and determining whether the first electrode material corresponds to a second cathode material candidate, when the first electrode material corresponds to the first cathode material candidate. The determining of whether the first electrode material corresponds to the first cathode material candidate includes generating cathode material feature information of the first electrode material from material feature information of the first electrode material, and determining whether the first electrode material corresponds to the first cathode material candidate, based on the cathode material feature information of the first electrode material. The material feature information includes chemical descriptor information and material characteristic information of an electrode material. The cathode material feature information includes composability information and cathode material core property information.

### STATEMENT REGARDING GOVERNMENT SPONSORED RESEARCH OR DEVELOPMENT

The present disclosure was developed in the task of a project to develop Development of a Next-Generation Solid Electrolyte Material Screening Platform: Integration of Deep Learning Generative Models and Bayesian Optimization Techniques (Project identification number: 1711179734, Project number: 2022R1F1A1074339, Ministry name: Ministry of Science and ICT, Project management organization name: National Research Foundation of Korea, Research project name: Individual basic research (Ministry of Science and ICT), project implementation organization name: Soongsil University, research period: 2023.03.01˜2024.02.29.)

### CROSS-REFERENCE TO RELATED APPLICATIONS

This application claims priority under 35 U.S.C. § 119 to Korean Patent Application No. 10-2024-0044610 filed on Apr. 2, 2024, in the Korean Intellectual Property Office, the disclosures of which are incorporated by reference herein in their entireties.

Meanwhile, in all the aspects of the inventive concept, there is no property interest in the government of the Republic of Korea.

### BACKGROUND

Embodiments of the present disclosure described herein relate to designing a battery cathode material with excellent performance using a machine learning technique. A conventional lithium-ion battery has been widely commercialized due to high energy density and long life. However, as there are growing concerns about the sustainability of the lithium-ion battery due to the scarcity and high cost of lithium resources. A sodium-ion battery which is emerging as an alternative to the lithium-ion battery has a similar mechanism to the lithium-ion battery and has low cost. However, a sodium-ion battery cathode material has limitations of a volume change and low structural stability between relatively lower specific gravity energy density and relatively larger charge and discharge period than the lithium-ion battery.

Thus, there is a demand in the industry for a technology for developing a sodium-ion battery cathode material with high energy density and high stability to overcome such a problem.

### SUMMARY

Embodiments of the present disclosure provide a technology for selecting a battery cathode material with excellent performance.

According to an embodiment, a method for designing a cathode material may include determining whether a first electrode material corresponds to a first cathode material candidate and determining whether the first electrode material corresponds to a second cathode material candidate, when the first electrode material corresponds to the first cathode material candidate. The determining of whether the first electrode material corresponds to the first cathode material candidate may include generating cathode material feature information of the first electrode material from material feature information of the first electrode material and determining whether the first electrode material corresponds to the first cathode material candidate, based on the cathode material feature information of the first electrode material. The material feature information may include chemical descriptor information and material characteristic information of an electrode material. The cathode material feature information may include composability information and cathode material core property information of the electrode material.

Furthermore, the first electrode material may belong to a sodium super ionic conductor (NASICON) material.

Furthermore, the chemical descriptor information may include at least one of elemental characteristic statistics information, electronic structure information, or ionic complex characteristic information of the electrode material.

Furthermore, the material characteristic information may include at least one of gravimetric capacity information, ion extraction degree information, or space group number information of the electrode material.

Furthermore, the composability information may include at least one of formation energy information or energy above hull information. The cathode material core property information may include a volume change.

Furthermore, the determining of whether the first electrode material corresponds to the first cathode material candidate may be performed using a plurality of machine learning models.

Furthermore, each of the plurality of machine learning models may independently generate the cathode material feature information of the first electrode material.

Furthermore, the determining of whether the first electrode material corresponds to the first cathode material candidate may include extracting first cathode material feature detailed information from a first machine learning model among the plurality of machine learning models and extracting second cathode material feature detailed information from a second machine learning model among the plurality of machine learning models. The first cathode material feature detailed information and the second cathode material feature detailed information may be different pieces of cathode material feature detailed information.

Furthermore, the determining of whether the first electrode material corresponds to the second cathode material candidate may include generating energy state information of the first electrode material and determining whether the first electrode material corresponds to the second cathode material candidate.

Furthermore, the generating of the energy state information of the first electrode material may be performed using a pre-trained graph neural network model.

Furthermore, the generating of the energy state information of the first electrode material may include performing density functional calculation for the first electrode material.

Furthermore, the determining of whether the first electrode material corresponds to the second cathode material candidate may include determining whether the energy state information of the first electrode material meets a predetermined criterion.

Furthermore, the predetermined criterion may be that an average voltage value of the first electrode material is greater than or equal to a predetermined value.

### BRIEF DESCRIPTION OF THE FIGURES

The above and other objects and features of the present disclosure will become apparent by describing in detail embodiments thereof with reference to the accompanying drawings.

FIG. 1 is a block diagram illustrating a configuration of a device for designing a cathode material according to an embodiment of the disclosed present disclosure.

FIG. 2 is a conceptual diagram illustrating a module and information for performing a method for designing a cathode material according to an aspect of the disclosed present disclosure.

FIG. 3 is a conceptual diagram illustrating a method for designing a cathode material according to an aspect of the disclosed present disclosure.

FIG. 4 is a chart illustrating feature importances of feature information of an electrode material according to an aspect of the disclosed present disclosure.

FIG. 5 is a chart illustrating performance for predicting cathode material feature information of an electrode material in a plurality of machine learning models according to an aspect of the disclosed present disclosure.

### DETAILED DESCRIPTION

The same reference denotations refer to the same components throughout the specification. This specification does not describe all elements of the embodiments, and overlaps between general contents or embodiments in the technical field to which the present disclosure pertains are omitted.

Furthermore, when a part “includes” a certain component, it means that other components may be further included rather than excluding other components unless specifically stated to the contrary.

The term “˜ unit” used in the specification may be a unit of processing at least one function or operation, which may refer to, for example, software, a field programmable gate array (FPGA), or a hardware component. A function provided from the “˜ unit” may be divided and performed by a plurality of components or may be integrated with other additional components. The “˜ unit” in the specification is not necessarily limited to software or hardware, which may be configured to be included in an addressable storage medium or to reproduce one or more processors. According to embodiments, a plurality of “˜ units” are able to be implemented as one component, or one “˜ unit” is able to include a plurality of components.

The terms first, second, etc. are used to distinguish one component from other components, and the component is not limited by these terms.

Singular expressions include plural expressions unless the context clearly indicates an exception.

In each step, the identification code is used for convenience of description, and the identification code does not describe the order of each step. Each of the steps may be performed out of the stated order unless the context clearly dictates the specific order.

Hereinafter, a description will be given of an operation principle and embodiments of the disclosed present disclosure with reference to the accompanying drawings.

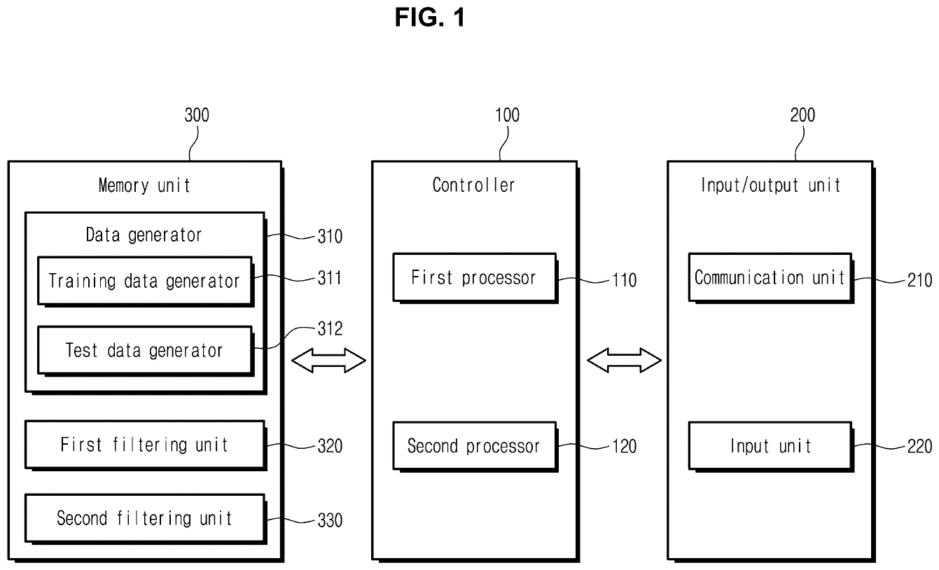

FIG. 1 is a block diagram illustrating a configuration of a device for designing a cathode material according to an embodiment of the disclosed present disclosure.

Referring to FIG. 1, the device for designing the cathode material according to the present disclosure may include a controller 100, an input/output unit 200, and a memory unit 300. As the components shown in FIG. 1 are not essential in implementing the device for designing the cathode material according to the present disclosure, data described on the specification may have components greater or less than the components listed above.

The controller 100 may be implemented with a memory (not shown) for storing an algorithm for controlling operations of the components in the device or data for a program implementing the algorithm and at least one processor (not shown) for performing the above-mentioned operation using the data stored in the memory. At this time, the memory and the processor may be implemented as separate chips, respectively. Alternatively, the memory and the processor may be implemented as a single chip.

The processor may include various logic circuits and operation circuits, may process data depending on a program provided from the memory, and may generate a control signal depending on the processed result.

The controller 100 according to some embodiments of the present disclosure may include one or more processors. Referring to FIG. 1, the controller 100 may include a first processor 110 and a second processor 120. In this case, the first processor 110 and the second processor 120 may be homogeneous or heterogeneous processors. In an embodiment, the first processor 110 may be a central processing unit (CPU), and the second processor 120 may be a graphics processing unit (GPU). In another embodiment, both the first processor 110 and the second processor 120 may be GPUs. In some embodiments, the first processor 110 and the second processor 120 may be implemented using a tensor processing unit (TPU), a neural processing unit (NPU), and/or the like. The controller 100 may include an additional processor necessary to drive the device for designing the cathode material, other than the first processor 110 and the second processor 120. Hereinafter, for convenience of description, the first processor 110 or the second processor 120 may be represented as a “processor”. The above-mentioned configuration of the controller 100 is only illustrative, and the configuration of the controller 100 is not limited thereto.

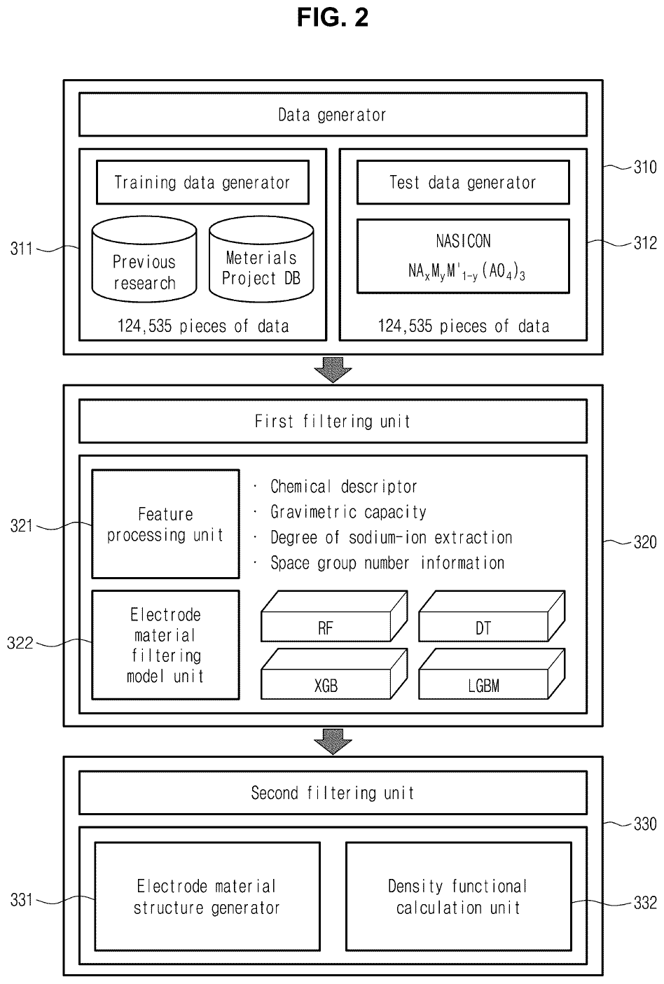

Furthermore, the controller 100 may control any one of the above-mentioned components or may combine and control a plurality of components among the above-mentioned components to implement various embodiments according to the present disclosure, which will be described with reference to FIGS. 2 to 5, on the device.

At least one component is added or deleted in response to performance of the components shown in FIG. 1. Furthermore, it may be easily understood to those skilled in the art that mutual positions of the components are able to change in response to the performance or structure of the system.

Meanwhile, each component shown in FIG. 1 refers to software and/or a hardware component such as a field programmable gate array (FPGA) and an application specific integrated circuit (ASIC).

Referring again to FIG. 1, a communication unit 210 according to some embodiments of the present disclosure may include one or more components capable of communicating with an external device and may include at least one of, for example, a wired communication module, a wireless communication module, or a short range communication module.

The wired communication module may include various cable communication modules, such as a universal serial bus (USB), a high definition multimedia interface (HDMI), a digital visual interface (DVI), recommended standard 232 (RS-232), power line communication, or a plain old telephone service (POTS), as well as various wired communication modules, such as a local area network (LAN) module, a wide area network (WAN) module, or a value added network (VAN) module.

The wireless communication module may include a wireless communication module which supports various wireless communication schemes such as global system for mobile communication (GSM), code division multiple access (CDMA), wideband CDMA (WCDMA), universal mobile telecommunication system (UMTS), time division multiple access (TDMA), long term evolution (LTE), 4th generation (4G), 5th generation (5G), or 6th generation (6G), other than a wireless-fidelity (Wi-Fi) module and a wireless broadband (WiBro) module.

The wireless communication module may include a wireless communication interface including an antenna and a transmitter for transmitting a Wi-Fi signal. Furthermore, the wireless communication module may further include a Wi-Fi signal conversion module for modulating a digital control signal output from the controller 100 via the wireless communication interface into a wireless signal in an analog form under control of the controller 100.

The wireless communication module may include a wireless communication interface including an antenna and a receiver for receiving a Wi-Fi signal. Furthermore, the wireless communication module may further include a Wi-Fi signal conversion module for demodulating a wireless signal in an analog form, which is received via the wireless communication interface, into a digital control signal.

The short range communication module may be for short range communication, which may support short range communication, using at least one of Bluetooth™, radio frequency identification (RFID), infrared data association (IrDA), ultra wideband (UWB), ZigBee, near field communication (NFC), wireless-fidelity (Wi-Fi), Wi-Fi Direct, and wireless universal serial bus (USB) technologies.

An input unit 220 may receive audio information (or signal), information including text or the like, and data from a network or a user, which may include at least one of at least one microphone and a user input unit. Data collected by the input unit 220 may be analyzed to be processed as a control command of the user.

The user input unit is to receive information from the user. When information is received via the user input unit, the controller 100 may control an operation of the device to correspond to the received information. Such a user input unit may include a hardware physical key (e.g., a button, a dome switch, a jog wheel, a jog switch, or the like located on at least one of the front, the rear, and the side of the device) and a software touch key. As an example, the touch key may be composed of a virtual key, a soft key, or a visual key displayed on a touch screen-type display unit through software processing and may be composed of a touch key disposed on a portion except for the touch screen. Meanwhile, the virtual key or the visual key is able to be displayed on the touch screen while having various forms, which may be composed of, for example, graphics, text, an icon, or a video, or any combination thereof.

The memory unit 300 may store data for supporting various functions of the device and a program for an operation of the controller 100, may store pieces of input/output data (e.g., a voice file, text, and the like), and may store a plurality of application programs or applications run in the device, pieces of data for an operation of the device, and instructions. At least some of such application programs may be downloaded from an external server through wireless communication.

The memory 300 may include at least one type of storage medium among a flash memory type memory, a hard disk type memory, a solid state disk (SSD) type memory, a silicon disk drive (SDD) type memory, a multimedia card micro type memory, a card type memory (e.g., a secure digital (SD) memory, an extreme digital (XD) memory or the like), a random access memory (RAM), a static RAM (SRAM), a read-only memory (ROM), an electrically erasable programmable ROM (EEPROM), a programmable ROM (RPOM), a magnetic memory, a magnetic disc, and an optical disc. Furthermore, the memory may be separated from the device, but may be a database connected in a wired or wireless manner.

Herein, the program may include a program instruction, a data file, a data structure, and the like independently or may include a combination thereof. The program may be designed and manufactured using a machine language code or a high-level language code. The program may be particularly designed to implement the above-mentioned method for designing the cathode material and may be implemented using various functions or definitions which are well known to those skilled in the computer software field and are available. The program for implementing the above-mentioned method for designing the cathode material may be recorded in a storage medium readable by the processor.

The memory may store a program which performs the above-mentioned operation and an operation which will be described below. The processor may execute the program stored in the memory. When the processor and the memory are plural in number, they are able to be integrated into one chip and are able to be provided at positions which are physically separated. The memory may include a volatile memory, such as a state RAM (SRAM) or a dynamic RAM (DRAM), for temporarily storing data. Furthermore, the memory may include a non-volatile memory, such as a ROM, an erasable programmable ROM (EPROM), an electrically erasable programmable ROM (EEPROM), for storing a control program and control data for a long time.

A function associated with artificial intelligence according to the present disclosure may operate by means of the processor and the memory. The processor may be composed of one processor or a plurality of processors. At this time, the one processor or the plurality of processors may be a universal processor, such as a central processing unit (CPU), an application processor (AP), or a digital signal processor (DSP), a dedicated graphics processor, such as a graphics processing unit (GPU) or a vision processing unit (VPU), or a dedicated artificial intelligence processor, such as a neural processing unit (NPU). The one processor or the plurality of processors may control to process input data, depending on a predefined operation rule or an artificial intelligence model stored in the memory. Alternatively, when the one processor or the plurality of processors are the dedicated artificial intelligence processor or the dedicated artificial intelligence processors, the dedicated artificial intelligence processor may be designed in a hardware structure specialized for processing a specific artificial intelligence model.

The machine learning or artificial intelligence model according to some embodiments of the present disclosure is characterized by being created through learning. Herein, being created through the learning means that the predefined operation rule or the artificial intelligence model set to perform a desired characteristic (or purpose) is made as a basic artificial intelligence model is trained using a plurality of pieces of training data by a learning algorithm. Such learning may be performed in the device itself in which the artificial intelligence model according to the present disclosure is executed and may be performed by means of a separate server and/or system. An example of the learning algorithm may be, but is not limited to, supervised learning, unsupervised learning, semi-supervised learning, or reinforcement learning.

The artificial intelligence model may be composed of a plurality of neural network layers. Each of the plurality of neural network layers may have a plurality of weight values and may perform neural network operation by means of operation between the result of operation of a previous layer and the plurality of weight values. The plurality of weight values of the plurality of neural network layers may be optimized by the result of training the artificial intelligence model. For example, the plurality of weight values may be updated such that a loss value or a cost value obtained by the artificial intelligence model during the training process is reduced or minimized. The artificial neural network may include a deep neural network (DNN). The artificial neural network may include, for example, but is not limited to, a convolutional neural network (CNN), a deep neural network (DNN), a recurrent neural network (RNN), a restricted Boltzmann machine (RMB), a deep belief network (DBN), a bidirectional recurrent deep neural network (BRDNN), deep Q-networks, or the like.

According to an embodiment of the present disclosure, the processor may execute the artificial intelligence model. The artificial intelligence may refer to a machine learning technology based on an artificial neural network, which allows a machine to simulate and train human biological neurons. The methodology of artificial intelligence may be divided into supervised learning in which the solution (output data) to the problem (input data) is determined as the input data and the output are provided together as training data, unsupervised learning in which the solution (output data) to the problem (input data) is not determined as only the input data is provided without the output data, and reinforcement learning which proceeds with learning in the direction of maximizing a reward, as the reward is given in an external environment whenever taking any action in a current state, according to a learning scheme. Furthermore, the methodology of artificial intelligence may be divided according to architecture which is the structure of a learning model. The architecture of a widely used deep learning technique may be divided into a convolutional neural network (CNN), a recurrent neural network (RNN), a transformer, generative adversarial networks (GAN), and the like.

The device for designing the cathode material, which is used in the device and system according to the present disclosure, may execute one or more artificial intelligence and/or machine learning models. The device for designing the cathode material may be implemented with one or a plurality of artificial intelligence and/or machine learning models. The artificial intelligence model or the machine learning model may be composed of a neural network (or an artificial neural network) and may include a statistical learning algorithm which mimics biological neurons, which is studied from machine learning and cognitive science. A neural network may refer to the overall model with a problem solving ability, as an artificial neuron (a node) forming a network through the combination of synapses changes synaptic coupling strength through learning. The neurons of the neural network may include a combination of weight values or biases. The neural network may include one or more layers, each of which is composed of one or more neurons or nodes. Illustratively, the device may include an input layer, a hidden layer, and an output layer. The neural network constituting the device may change the weight values of the neurons through learning to infer a result (an output) to be predicted from any input.

The processor may generate a neural network, may train or learn the neural network, may perform calculation based on the received input data and generate an information signal based on the performed result, or may retrain the neural network. Models of the neural network may include, but are not limited to, various types of models, such as a convolution neural network (CNN), such as GoogleNet, AlexNet, or VGG Network, a region with convolution neural network (R-CNN), a region proposal network (RPN), a recurrent neural network (RNN), a stacking-based deep neural network (S-DNN), a state-space dynamic neural network (S-SDNN), a deconvolution network, a deep belief network (DBN), a restricted Boltzman machine (RBM), a fully convolutional network, a long short-term memory (LSTM) network, and a classification network. The processor may include one or more processors for performing calculation according to the models of the neural network. For example, the neural network may include a deep neural network.

Referring again to FIG. 1, the memory 300 according to some of the present disclosure may include a data generator 310, a first filtering unit 320, and/or a second filtering unit 330.

The data generator 310 according to some of the present disclosure may be a software module, a hardware module, and/or a combination thereof, which are/is for generating data used in the method for designing the cathode material. In an embodiment, the data generated by the data generator 310 may include training data and test data provided to a machine learning model, which are used in the method for designing the cathode material. In some embodiments, the data generator 310 may access a database outside the device for designing the cathode material to generate the training data and the test data. Furthermore, in an embodiment, the data generator 310 may generate the training data and the test data from different databases.

Referring again to FIG. 1, the data generator 310 according to some of the present disclosure may include a training data generator 311 and a test data generator 312.

In some embodiments, the training data generator 311 may be a software module, a hardware module, and/or a combination thereof, which are/is for generating data for training one or more machine learning models, which is used in the method for designing the cathode material according to the present disclosure. In an embodiment, the training data generated by the training data generator 311 may include information about an electrode material usable as the cathode material. In detail, the information about the electrode material may include material feature information and cathode material feature information of the electrode material. In this case, the material feature information may include chemical descriptor information and material characteristic information of the electrode material. Furthermore, the cathode material feature information may include composability information and cathode material core property information of the electrode material.

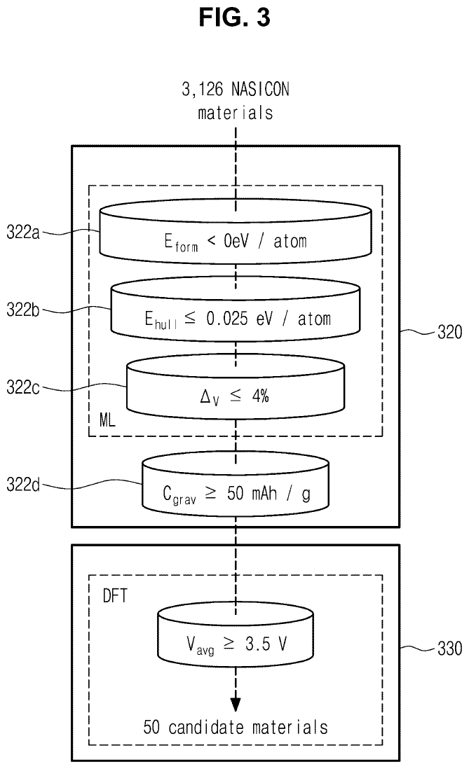

In some embodiment, the test data generator 312 may be a software module, a hardware module, and/or a combination thereof, which are/is for generating input data for determining whether a specific electrode material corresponds to a cathode material candidate, depending on the method for designing the cathode material according to the present disclosure. In an embodiment, the test data generated by the test data generator 312 may include the information about the electrode material. In an embodiment, the information about the electrode material included in the test data may include only material feature information of the electrode material. Although will be described below with reference to FIG. 3, the test data according to the present disclosure may be generated based on material characteristic information of a plurality of electrode materials included in a specific material group. In other words, all of electrode materials associated with pieces of electrode material information included in the test data may be included in the same material group. A description will be given in detail below of the training data and the test data with reference to FIG. 3.

Referring again to FIG. 1, the memory unit 300 according to some of the present disclosure may include the first filtering unit 320.

In some embodiments, the first filtering unit 320 may be a software module, a hardware module, and/or a combination thereof, which are/is for filtering a related electrode material based on input electrode material feature information. Preferably, the electrode material feature information input to the first filtering unit 320 may be the training data generated by the training data generator 311 and/or the test data generated by the test data generator 312. In an embodiment, the first filtering unit 320 may be implemented using one or more artificial intelligence and/or machine learning modules. Although will be described in detail with reference to FIGS. 2 and 3, one or more artificial intelligence and/or machine learning modules included in the first filtering unit 320 may be independently driven. Furthermore, as the one or more artificial intelligence and/or machine learning modules included in the first filtering unit 320 are connected in series with each other, electrode material feature information about the electrode material, which is filtered by one artificial intelligence and/or machine learning module, is input to the machine learning module located at a rear stage. In another embodiment, as the one or more artificial intelligence and/or machine learning modules included in the first filtering unit 320 are connected in parallel with each other, each of the machine learning and/or artificial intelligence modules may independently process the same electrode material feature information. In this case, the machine learning and/or artificial intelligence module may be used in an integrated manner using an ensemble technique. Alternatively, the plurality of machine learning and/or artificial intelligence modules may be used in a manner which extracts one piece of previously specified electrode material feature detailed information (or although will be described below, cathode material feature detailed information included in cathode material feature information included in electrode material feature information) from each of the machine learning and/or artificial intelligence modules.

Furthermore, in some embodiments, the first filtering unit 320 may additionally include sub-modules for constructing the machine learning and/or artificial intelligence module. In some embodiments, such sub-modules may include modules for loss function calculation, weight value adjustment, and learning rate adjustment for learning of the machine learning and/or artificial intelligence modules included in the first filtering unit 320, an optimizer, a feature processing unit for processing feature information, and/or the like.

In an embodiment, the feature processing unit included in the first filtering unit 320 may include a software module, a hardware module, and/or a combination thereof, which are/is for calculating feature importances of one or more pieces of feature information included in the electrode material information and determining a feature to be included in the training data based on the calculated feature importances. Preferably, the feature processing unit may calculate feature importances for a plurality of pieces of feature information included in the electrode material information and may determine feature information to be included in the training data based on the calculated feature importances. In detail, the feature processing unit may remove feature information, the feature importance of which is lower than a predetermined level, from the training data.

Referring again to FIG. 1, the memory unit 300 according to some of the present disclosure may include the second filtering unit 330. In an embodiment, the second filtering unit 330 may be a software module, a hardware module, and/or a combination thereof, which are/is for filtering the electrode materials primarily filtered through the first filtering unit 320 again. Preferably, the electrode materials filtered through the second filtering unit 330 may be determined as a cathode material candidate by the method for designing the cathode material according to the present disclosure.

Like the first filtering unit 320, the second filtering unit 330 may include one or more machine learning and/or artificial intelligence modules. Preferably, the one or more machine learning and/or artificial intelligence modules included in the second filtering unit 330 may be modules for determining a structure of the electrode material. More preferably, the one or more machine learning and/or artificial intelligence modules included in the second filtering unit 330 may be pre-trained to determine the most stable structure of the electrode material. In this case, in an example, the one or more machine learning and/or artificial intelligence modules included in the second filtering unit 330 may use a graph neural network. Preferably, materials graph neural networks with three-body interactions (M3GNet) may be used.

Furthermore, the second filtering unit 330 may include a software module, a hardware module, and/or a combination thereof, which are/is for calculating energy state information of the electrode material to filter the electrode material. In an embodiment, the calculated information about the energy state may be information about an average voltage predicted from the electrode material. Furthermore, in an embodiment, the calculation of an energy level of the electrode material according to a density functional theory may be performed by at least a part of the second filtering unit 330 to calculate the energy state information. A description will be given in detail of it with reference to FIGS. 2 and 3.

FIG. 2 is a conceptual diagram illustrating a module and information for performing a method for designing a cathode material according to an aspect of the disclosed present disclosure.

Referring to FIG. 2, a data generator 310 according to some of the present disclosure may include a training data generator 311 and a test data generator 312. As described above with reference to FIG. 1, the data generator 310 in the present disclosure may generate training data and/or test data to include feature information processed by a feature processing unit 321 included in a first filtering unit 320.

The training data generator 311 according to some of the present disclosure may be a software module, a hardware module, and/or a combination thereof, which are/is for generating data for training the first filtering unit 320 and/or a second filtering unit 330. In an embodiment, the training data may be generated from data for characteristics for a charge/discharge chemical formula, an operating ion, an average voltage, a volume change, capacity, formation energy, and energy above hull from an electrode database (DB) used in conventional studies and a materials project (MP) DB. In some embodiments, the training data may be generated by removing an outlier value from characteristic information obtained from the electrode DB and the MP DB. In an embodiment, the outlier value may be information in which the average voltage is negative. In some embodiments, the training data may include chemical descriptor information, material characteristic information, composability information, and/or cathode material core property information of an electrode material. In an embodiment, the chemical descriptor information may include elemental characteristic statistics information, electronic structure information, and/or ionic complex characteristic information of the electrode material. In another embodiment, the material characteristic information may include information closely associated with battery performance, such as gravimetric capacity information, ion extraction degree information, and/or space group number information of the electrode material. In another embodiment, the composability information may include formation energy information and/or energy above hull information, and the cathode material core property information may include volume change information and/or average voltage information.

In some embodiments of the present disclosure, the chemical descriptor information of the electrode material may be a set of numbers or signs quantitatively indicating structural, physical, and chemical properties of a chemical substance. In an embodiment, the chemical descriptor may be used to convert a characteristic of a chemical substance into a form understandable by a computer, search a chemical database, predict properties of a compound, study chemical reactivity, or the like. As an example, the chemical descriptor may include a structural descriptor and/or a physical and chemical descriptor. Preferably, the elemental characteristic statistics information, the electronic structure information, and/or the ionic complex characteristic information of the electrode material in the chemical descriptor information may be included in the training data and the test data according to some of the present disclosure.

In some embodiments of the present disclosure, the material characteristic information may refer to additional information for describing a characteristic of the electrode material, which differentiates from the chemical descriptor. In an embodiment, the material characteristic information may be information closely associated with the performance of a battery. In some embodiments, the gravimetric capacity information, ion extraction degree information, and/or space group number information of the electrode material in such material characteristic information may be included in the training data and the test data according to the present disclosure.

Referring again to FIG. 1, the test data according to some of the present disclosure may be input data for determining whether a specific electrode material corresponds to a cathode material candidate. In an embodiment, the test data generated by the test data generator 312 may include information about the electrode material. Preferably, the information about the electrode material, which is included in the test data, may include material feature information of the electrode material. Although will be described below with reference to FIG. 3, the test data according to the present disclosure may be generated based on material characteristic information of a plurality of electrode materials included in a specific material group. In other words, all of electrode materials associated with specific electrode material information included in the test data may be included in the same material group.

Preferably, the material group associated with the electrode material information included in the test data according to the present disclosure may be a sodium super ionic conductor (NASICON), which may be a material group including materials with a crystal structure capable of very efficiently delivering a sodium ion. In an embodiment, the test data generator 312 may generate electrode material information (or material feature information) for all chemically feasible NASICON materials and may determine it as test data.

In detail, the test data generator 312 according to embodiment of the present disclosure may generate a test dataset, based on Chemical Formula A below.

NaₓVM_{y}M′_{1-y}(AO₄)₃ [Chemical Formula A]

In an embodiment, the test data generator 312 may generate a test dataset including a compound considering 27 elements at M and M′ positions and 6 elements at position A, depending on Chemical Formula A above. Preferably, the test data generator 312 may use one of NASICON cathode materials widely studied, Na₃V₂(PO₄)₃, as a parent structure. Furthermore, the test data generator 312 may consider total 27 elements, such as V, Al, Ca, Cd, Co, Cr, Cu, Fe, Ga, Ge, Hf, In, La, Lu, Mg, Mn, Nb, Ni, Sb, Sc, Sn, Ta, Ti, Y, Yb, Zn, and Zr, as doping elements at positions M and M′ and may consider 6 elements, such as Si, S, Se, As, Mo, and P, at position A. When generating the test dataset, the test data generator 312 may consider a plurality of doping rates at positions M and M′. Preferably, the doping rates may be 0.5:0.5, 0.333:0.667, and 0.667:0.333. Furthermore, the test data generator 312 may adjust the number of sodium ions such that the sum of all oxidation numbers is 0. Preferably, the number of the sodium ions may be 0 to 4. More preferably, the test data generator 312 may remove a NASICON compound, the number of sodium ions of which is greater than or equal to 5, from the test dataset.

Referring again to FIG. 2, the first filtering unit 320 according to some of the present disclosure may include the feature processing unit 321 and/or an electrode material filtering model unit 322.

In some embodiments, the first filtering unit 320 may be a software module, a hardware module, and/or a combination thereof, which are/is for filtering a related electrode material based on input electrode material feature information. In some embodiments, the first filtering unit 320 may filter an electrode material using one or more artificial intelligence models. At this time, the data filtered by the first filtering unit 320 may include training data and test data generated by the data generator 310.

Although not illustrated in FIG. 2, the first filtering unit 320 may additionally include sub-modules for constructing the machine learning and/or artificial intelligence module. In some embodiments, such sub-modules may include modules for loss function calculation, weight value adjustment, and learning rate adjustment for learning of the machine learning and/or artificial intelligence modules included in the first filtering unit 320, an optimizer, the feature processing unit 321 for processing feature information, and/or the like.

The feature processing unit 321 according to some of the present disclosure may include a software module, a hardware module, and/or a combination thereof, which are/is for calculating feature importances of one or more pieces of feature information included in the electrode material information and determining a feature to be included in the training data based on the calculated feature importances. Preferably, the feature processing unit 321 may calculate feature importances for a plurality of pieces of feature information included in the electrode material information and may determine feature information to be included in the training data based on the calculated feature importances. In detail, the feature processing unit 321 may remove feature importance, the feature importance of which is lower than a predetermined level, from the training data. As shown in FIG. 2, the feature information processed by the feature processing unit 321 may include chemical descriptor information, gravimetric capacity information, ion extraction degree information, space group number information, and/or the like.

The gravimetric capacity information, the ion extraction degree information, and/or the space group number information corresponding to the material characteristic information among the pieces of feature information according to some of the present disclosure may be characteristics with a close association with battery performance. For example, the gravimetric capacity information may be closely associated with energy density of the entire battery. Because the ion extraction degree information indicates how many operating ions are extracted from the anode material during charging, it may be closely associated with a battery volume change and an average voltage. Furthermore, the space group number information may be included in the feature information to distinguish a polymorph structure.

The electrode material filtering model unit 322 according to some of the present disclosure may include one or more machine learning and/or artificial intelligence models. In some embodiments, the electrode material filtering model unit 322 may be constructed using two or more different types of machine learning and/or artificial intelligence models. In an embodiment, the machine learning and/or artificial intelligence model included in the electrode material filtering model unit 322 may use a neural network-based deep learning model. In another embodiment, the machine learning and/or artificial intelligence model included in the electrode material filtering model unit 322 may use a decision tree and a decision tree-based ensemble learning method, such as random forest (RF), decision tree (DT), XGBoost, and/or light gradient boosting machine (LGBM). In a preferable embodiment, a plurality of machine learning and/or artificial intelligence models included in the electrode material filtering model unit 322 may independently operate to filter an electrode material from the input training data and/or test data. In an embodiment, the plurality of machine learning and/or artificial intelligence models included in the electrode material filtering model unit 322 may be configured in parallel. In another embodiment, as the plurality of machine learning and/or artificial intelligence models included in the electrode material filtering model unit 322 is configured in series, the filtering of the machine learning and/or artificial intelligence model located at a rear stage may be performed based on the filtering result of the machine learning and/or artificial intelligence model located at a front stage.

In some embodiments, the plurality of machine learning and/or artificial intelligence models included in the electrode material filtering model unit 322 may generate cathode material feature information of the electrode material as the output from the input data to filter the electrode material. At this time, in some embodiments, the cathode material feature information may include one or more pieces of cathode material feature detailed information. In some embodiments, the plurality of machine learning and/or artificial intelligence models included in the electrode material filtering model unit 322 may generate composability information and/or cathode material core property information as the cathode material feature information. In some embodiments, the composability information of the electrode material may include formation energy information and/or energy above hull information. Furthermore, the cathode material core property information may include volume change information, average voltage information, and gravimetric capacity information. As described above, the formation energy information, the energy above hull information, the volume change information, the average voltage information, the gravimetric capacity information, and/or the like may correspond to the cathode material feature detailed information. Preferably, the electrode material filtering model unit 322 according to some of the present disclosure may extract different cathode material feature detailed information from each of the plurality of machine learning and/or artificial intelligence models and may determine it as the cathode material feature information. For example, as shown in Table 1 below, when a random forest model, a decision tree model, an XGBoost model, and an LGBM model are independently present in the electrode material filtering model unit 322, the electrode material filtering model unit 322 may extract formation energy information from the random forest model, may extract average voltage information from the LGBM model, may extract volume change information from the XGBoost model, and may extract energy above hull information from the decision tree model. Preferably, when there is a machine learning and/or artificial intelligence model with the best prediction result for specific cathode material feature detailed information, the electrode material filtering model unit 322 may allow the machine learning and/or artificial intelligence model to extract the cathode material feature detailed information.

Referring again to FIG. 2, the second filtering unit 330 according to some of the present disclosure may be a software module, a hardware module, and/or a combination thereof, which are/is for filtering the electrode materials primarily filtered by means of the first filtering unit 320 again. As shown in FIG. 2, the second filtering unit 330 according to an embodiment of the present disclosure may include an electrode material structure generator 331 and a density functional calculation unit 332.

The electrode material structure generator 331 according to some of the present disclosure may include a software module, a hardware module, and/or a combination thereof, which are/is for generating a crystal structure of the electrode material to generate energy state information of the electrode material. In some embodiments, the crystal structure of the electrode material, which is generated by means of the electrode material structure generator 331, may be the most stable structure of the electrode material. In some embodiments, the electrode material structure generator 331 may generate the crystal structure of the electrode material using a neural network-based machine learning and/or artificial intelligence module. In an embodiment, the electrode material structure generator 331 may be constructed using a graph neural network model. As an example, the electrode material structure generator 331 may generate the most stable crystal structure of the electrode material using an M3GNet model. At this time, the generated most stable crystal structure of the electrode material may include the most stable discharge structure and the most stable charge structure.

In some embodiments, the density functional calculation unit 332 may include a software module, a hardware module, and/or a combination thereof, which are/is for calculating an energy level of the electrode material according to a density functional theory (DFT) to generate energy state information of the electrode material generated by means of the electrode material structure generator 331. In an embodiment, the density functional calculation unit 332 may perform DFT calculation for energy density, an average voltage, and/or a volume change for the crystal structure of the electrode material.

In some embodiments, the second filtering unit 330 may filter the electrode material, based on the energy state information of the electrode material generated by the density functional calculation unit 332. Preferably, only when the energy state information of the electrode material meets a predetermined criterion, the second filtering unit 330 may determine the electrode material as a second cathode material candidate. In an embodiment, the predetermined criterion for determining the electrode material as the second cathode material candidate may be determined according to whether the average voltage is greater than or equal to a predetermined level. In another embodiment, the predetermined criterion for determining the electrode material as the second cathode material candidate may be determined according to whether a relationship between the volume change and the average voltage according to the DFT calculation meets the predetermined criterion. For example, when having an average voltage higher than a volume change level, the electrode material may be determined as the second cathode material candidate.

FIG. 3 is a conceptual diagram illustrating a method for designing a cathode material according to an aspect of the disclosed present disclosure.

Referring to FIG. 3, the method for designing a cathode material according to some of the present disclosure may be to determine whether an electrode material corresponds to a cathode material candidate through a first filtering unit 320 and a second filtering unit 330 sequentially for input data.

In some embodiments, as shown in FIG. 3, the first filtering unit 320 may include a software module, a hardware module, and/or a combination thereof, which are/is for predicting cathode material feature information of the electrode material and determining whether the electrode material corresponds to a first cathode material candidate depending on whether the predicted cathode material feature information meets a predetermined criterion.

In an embodiment, although not illustrated in FIG. 3, the first filtering unit 320 may generate the cathode material feature information of the electrode material from material feature information of the electrode material which is the input data. In an embodiment, the material feature information may include chemical descriptor information and/or material characteristic information of the electrode material. In another embodiment, the cathode material feature information may include composability information and cathode material core property information of the electrode material. As shown in FIG. 3, the first filtering unit 320 may include a plurality of machine learning models 322a, 322b, and 322c. In an embodiment, each of the plurality of machine learning models 322a, 322b, and 322c may generate cathode material feature information of the electrode material from the material feature information of the electrode material. In an embodiment, the filtering of the electrode material by the first filtering unit 320 may be performed, according to whether each of pieces of cathode material feature information included in the generated cathode material feature information of the electrode material meets a predetermined criterion. In an embodiment, each of the plurality of machine learning models 322a, 322b, and 322c included in the first filtering unit 320 may filter one piece of cathode material feature detailed information. For example, the first machine learning model 322a may only filter formation energy information which is one of pieces of composability information of the electrode material in the generated cathode material feature information. The second machine learning model 322b may only filter energy above hull. The third machine learning model 322c may only filter a volume change. In an embodiment, a detailed module 322d for filtering gravimetric capacity may be implemented as a machine learning and/or artificial intelligence module and may be implemented to filter electrode materials, gravimetric capacity of which meets the predetermined criterion, using logic except for machine learning and/or artificial intelligence. In an example, as shown in FIG. 3, preferably, the electrode material with cathode material feature information in which the formation energy is negative, the energy above hull is 0.025 eV/atom, the volume change is less than or equal to 4%, and the gravimetric capacity is greater than or equal to 50 mAh/g may be determined as a first cathode material candidate by the first filtering unit 320. The filtering by the plurality of machine learning models 322a, 322b, and 322c and the detailed module 322d is performed in series in FIG. 3, but filtering by a plurality of machine learning models and detailed modules are not limited to such a scheme. For example, the plurality of machine learning models and the detailed modules may perform filtering in parallel and may determine an electrode material meeting all the filtered results or meeting a predetermined number of filtered results as the first cathode material candidate. Furthermore, the number of machine learning models included in the first filtering unit 320 is not limited to that shown in FIG. 3. In other words, the number of machine learning models included in the first filtering unit 320 may change to be different from that shown in FIG. 3.

Referring again to FIG. 3, the second filtering unit 330 according to some of the present disclosure may be a software module, a hardware module, and/or a combination thereof, which are/is for filtering the electrode materials primarily filtered by means of the first filtering unit 320 again. Preferably, the electrode materials filtered by means of the second filtering unit 330 may be determined as a cathode material candidate by the method for designing the cathode material according to the present disclosure.

In some embodiments, the second filtering unit 330 may determine whether at least some of characteristics of the electrode material meet a predetermined criterion by means of DFT calculation, thus determining the electrode materials as the second cathode material candidate of the electrode material. In an embodiment, the second filtering unit 330 may determine a structure of the electrode material before performing the DFT calculation. Preferably, at least some of modules included in the second filtering unit 330 may be for determining the structure of the electrode material. More preferably, the second filtering unit 330 may use a machine learning and/or artificial intelligence-based module for determining the structure of the electrode material. For example, the second filtering unit 330 may use M3GNet to generate a crystal structure of the electrode material. At this time, the determined structure of the electrode material may be the most stable structure of the electrode material.

In some embodiments, the second filtering unit 330 may determine energy state information of the electrode material by means of the DFT calculation. In an embodiment, the energy state information may include energy density, an average voltage value, and/or a volume change of the electrode material. In an embodiment, when a relationship between at least two of the energy density, the average voltage value, and the volume change generated by means of the DFT calculation meets the predetermined criterion, the electrode material may be determined as the second cathode material candidate. For example, when the volume change compared to the average voltage value is greater than or equal to a predetermined value, the electrode material may be determined as the second cathode material candidate. In another embodiment, when the average voltage value generated by means of the DFT calculation is greater than or equal to the predetermined value, the electrode material may be determined as the second cathode material candidate.

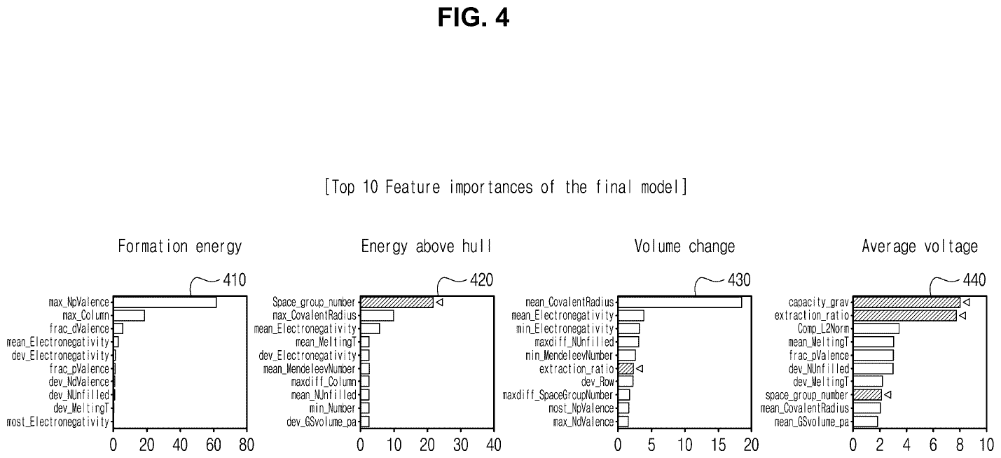

FIG. 4 is a chart illustrating feature importances of feature information of an electrode material according to an aspect of the disclosed present disclosure.

Charts disclosed in FIG. 4 may be charts for arranging feature importance values, in each of a plurality of machine learning models for extracting cathode material feature information. In an embodiment, the charts shown in FIG. 4 may be a formation energy feature importance chart 410, an energy above hull feature importance chart 420, a volume change feature importance chart 430, and an average voltage feature importance chart 440.

Referring to the charts shown in FIG. 4, it may be verified that features included in material characteristic information have the high feature importances. In an embodiment, it may be verified that space group number information has the high importance to extract energy above hull feature information, that the ion extraction degree information has the high importance to extract volume change feature information, and that the gravimetric capacity information, the ion extraction degree information and the space group number information have the high importances to extract average voltage information. Thus, when a plurality of machine learning and/or artificial intelligence models for extracting cathode material feature information, which are included in a first filtering unit 320 and/or a second filtering unit 330, are trained and estimated, as pieces of material characteristic information are added, the filtering performance of the first filtering unit 320 and/or the second filtering unit 330 may be greatly improved.

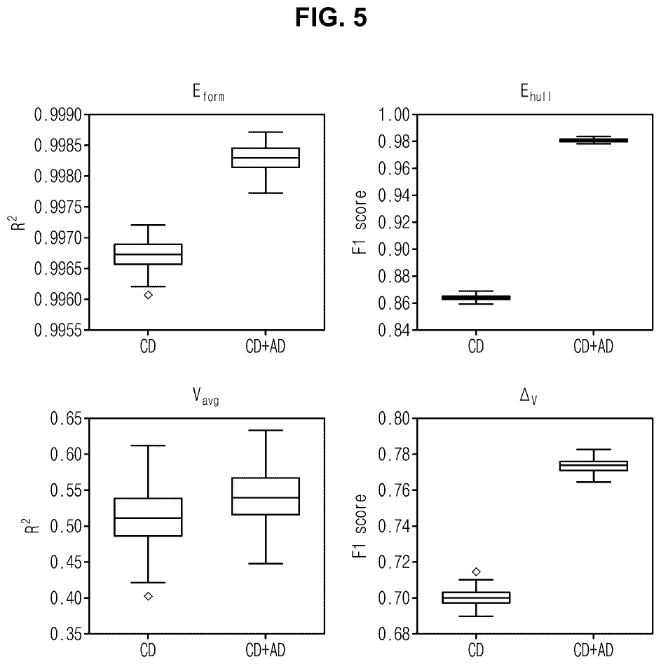

FIG. 5 is a chart illustrating performance for predicting cathode material feature information of an electrode material in a plurality of machine learning models according to an aspect of the disclosed present disclosure.

Charts shown in FIG. 5 may be charts for expressing performance indexes for a test dataset of a plurality of machine learning models for extracting cathode material feature information. Referring to the charts shown in FIG. 5, it may be verified that there is larger improvement in an R-square value and an F1-score value when extracting cathode material feature information by using both of chemical descriptor information and material feature information (CA+AD), than when extracting cathode material feature information by only the chemical descriptor information CD. Thus, when a plurality of machine learning and/or artificial intelligence models for extracting cathode material feature information, which are included in a first filtering unit 320 and/or a second filtering unit 330, are trained and estimated, as material characteristic information according to the present disclosure is added to feature information, the filtering performance of the first filtering unit 320 and/or the second filtering unit 330 may be greatly improved.

According to an aspect of the disclosed present disclosure, excellent battery cathode material candidates may be quickly searched.

As described above, the disclosed embodiments have been described with reference to the accompanying drawings. It will be obvious to those of ordinary skill in the art that the present disclosure may be practiced in different forms from the embodiments as described above without changing the technical idea or essential features of the present disclosure. The disclosed embodiments are illustrative and should not be construed as limiting.

What is claimed is:

1. A method for designing a cathode material, the method comprising:
determining whether a first electrode material corresponds to a first cathode material candidate; and
determining whether the first electrode material corresponds to a second cathode material candidate, when the first electrode material corresponds to the first cathode material candidate,
wherein the determining of whether the first electrode material corresponds to the first cathode material candidate includes:
generating cathode material feature information of the first electrode material from material feature information of the first electrode material; and
determining whether the first electrode material corresponds to the first cathode material candidate, based on the cathode material feature information of the first electrode material,
wherein the material feature information includes chemical descriptor information and material characteristic information of an electrode material, and
wherein the cathode material feature information includes composability information and cathode material core property information of the electrode material.

2. The method of claim 1, wherein the first electrode material belongs to a sodium super ionic conductor (NASICON) material.

3. The method of claim 2, wherein the chemical descriptor information includes at least one of elemental characteristic statistics information, electronic structure information, or ionic complex characteristic information of the electrode material.

4. The method of claim 2, wherein the material characteristic information includes at least one of gravimetric capacity information, ion extraction degree information, or space group number information of the electrode material.

5. The method of claim 2, wherein the composability information includes at least one of formation energy information or energy above hull information, and
wherein the cathode material core property information includes a volume change.

6. The method of claim 1, wherein the determining of whether the first electrode material corresponds to the first cathode material candidate is performed using a plurality of machine learning models.

7. The method of claim 6, wherein each of the plurality of machine learning models independently generates the cathode material feature information of the first electrode material.

8. The method of claim 7, wherein the determining of whether the first electrode material corresponds to the first cathode material candidate includes:
extracting first cathode material feature detailed information from a first machine learning model among the plurality of machine learning models; and
extracting second cathode material feature detailed information from a second machine learning model among the plurality of machine learning models, and
wherein the first cathode material feature detailed information and the second cathode material feature detailed information are different pieces of cathode material feature detailed information.

9. The method of claim 1, wherein the determining of whether the first electrode material corresponds to the second cathode material candidate includes:
generating energy state information of the first electrode material; and
determining whether the first electrode material corresponds to the second cathode material candidate.

10. The method of claim 9, wherein the generating of the energy state information of the first electrode material is performed using a pre-trained graph neural network model.

11. The method of claim 9, wherein the generating of the energy state information of the first electrode material includes:
performing density functional calculation for the first electrode material.

12. The method of claim 9, wherein the determining of whether the first electrode material corresponds to the second cathode material candidate includes:
determining whether the energy state information of the first electrode material meets a predetermined criterion.

13. The method of claim 12, wherein the predetermined criterion is that an average voltage value of the first electrode material is greater than or equal to a predetermined value.

14. A device for designing a cathode material to execute a method for designing the cathode material according to claim 1.

15. A computer-readable storage medium storing a computer program for performing a method for designing a cathode material according to claim 1.
